(19) United States
(12) Patent Application Publication
(10) Pub. No.: US 2006/0019237 A1
(43) Pub. Date: Jan. 26, 2006

(54) In vitro wound healing assay and device

(75) Inventors: Christine E. Pullar, Sacramento, CA (US); R. Rivkah Isseroff, Sacramento, CA (US)

Correspondence Address: QUINE INTELLECTUAL PROPERTY LAW GROUP, P.C., P O BOX 458, ALAMEDA, CA, 94501, US

(73) Assignee: The Regents of the University of California, Oakland, CA (US)

(21) Appl. No.: 11/185,434

(22) Filed: Jul. 19, 2005

Related U.S. Application Data
(60) Provisional application No. 60/590,235, filed on Jul. 20, 2004.

(51) Int. Cl. C12Q 1/00; C12Q 1/68; C12N 5/08

(52) U.S. Cl. 435/4; 435/371; 435/6

(57) ABSTRACT

Devices for creating model wounds in vitro are provided, along with kits and systems including the devices. Methods for making such devices and for modeling wounds are also provided.

### CROSS-REFERENCE TO RELATED APPLICATIONS

This application is a non-provisional utility patent application claiming priority to and benefit of the following prior provisional patent applications: U.S. Ser. No. 60/590,235, filed Jul. 20, 2004, entitled “IN VITRO WOUND HEALING ASSAY AND DEVICE” by Christine E. Pullar and R. Rivkah Isseroff, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with government support under Grant No. 1 K01 AR48827-01A1 from the National Institutes of Health National Institute of Arthritis and Musculoskeletal and Skin Diseases. The government may have certain rights to this invention.

### FIELD OF THE INVENTION

This invention relates to devices for creating model wounds in vitro and to methods for modeling wounds and wound healing in vitro.

### BACKGROUND OF THE INVENTION

In vitro wound healing assays in which a model wound is created in a layer of cells in tissue culture are used, for example, to monitor the migration, directional migration, and/or proliferation of various cell types under different culture conditions. Such assays typically involve growing a confluent cell layer, e.g., in a culture dish, and then destroying or displacing some of the cells to leave a gap or “wound”. The model wound is then monitored, e.g., by microscopic and/or photographic inspection, over time as cells migrate and/or divide to fill in and “heal” the damaged area.

Currently, the most widespread method of disrupting the cell layer to create a model wound involves scratching a path through the cell layer with a pointed object (e.g., a pipette tip, needle, cell scraper, or razor blade). See, e.g., U.S. Pat. No. 6,309,818 to Malinda et al. entitled “Scratch wound assay device.” However, this method typically results in variability in width along the length of the wound, variability in width between wounds, and/or damage to the cells at the edge of the wound (which can, e.g., prevent cell migration into the wound site and healing). In addition, although this method is simple, it can be costly in terms of time and reagents (e.g., cells and culture media), since a high percentage of experiments are unusable due to failure of the wounds to heal (due, e.g., to damage to cells at the edge of the wound).

Published U.S. patent application No. 20020182591 by Giaever et al. entitled “Electrical wounding assay for cells in vitro” describes an alternative method for wounding a cell layer. In this method, an electrical signal is used to kill a small patch of cells, creating a wound. This method, however, requires appropriate equipment, and the size and/or shape of the wound can not be conveniently varied.

Among other benefits, the present invention provides methods and devices that overcome the above noted difficulties (e.g., variability in wound width and damage to cells adjacent to the wound). A complete understanding of the invention will be obtained upon review of the following.

### SUMMARY OF THE INVENTION

The present invention provides methods and devices for simply and reproducibly creating model wounds in vitro, e.g., wounds of varying, preselected sizes in cell, tissue, or organ culture. Related kits, systems, and methods of producing such devices are also described.

A first general class of embodiments provides a device for modeling wounds in vitro. The device includes a first cell growth substrate and a second cell growth substrate. The second cell growth substrate is disposed on the first cell growth substrate, and at least a portion of the second cell growth substrate is removable from the first cell growth substrate.

The first cell growth substrate can be, e.g., a tissue culture dish, a well of a multiwell tissue culture plate, or a tissue culture slide or similar planar surface. In a preferred class of embodiments, the first cell growth substrate comprises a biocompatible material. For example, the first cell growth substrate can comprise polystyrene, polypropylene, or polycarbonate. In one embodiment, the first cell growth substrate comprises glass, e.g., glass coated with a biocompatible material.

The second cell growth substrate preferably comprises a biocompatible material. For example, in one class of embodiments, the second cell growth substrate comprises or consists of a plastic or polymer film.

In one class of embodiments, the entire second cell growth substrate is removable from the first cell growth substrate. In another class of embodiments, a first portion of the second cell growth substrate is removable from the first cell growth substrate. A second portion of the second cell growth substrate remains disposed on the first cell growth substrate upon removal of the first portion. In certain embodiments, the second cell growth substrate comprises a tearable material. In one class of example embodiments, the second cell growth substrate comprises a plastic or polymer film that is perforated in a preselected pattern. The pattern delineates at least one portion of the second cell growth substrate to be removed.

Depending, e.g., on the configuration of the portion(s) of the second cell growth substrate to be removed, the device can be used to create one or more model wounds, of the same or different sizes, in essentially any desired pattern. For example, in one class of embodiments, two or more portions of the second cell growth substrate are independently removable from the first cell growth substrate.

As noted, the device is useful for creating model wounds, e.g., by disrupting a confluent layer of cells, a cell layer in a tissue or organ culture, or the like. Thus, in one class of embodiments, the device includes a confluent layer of cells disposed on the second cell growth substrate (and, in certain embodiments, also on the first cell growth substrate), whereby removal of at least a portion of the second cell growth substrate from the first cell growth substrate creates a model wound in the confluent layer of cells. In a related class of embodiments, the device comprises a confluent layer of cells disposed on the second cell growth substrate (and, in certain embodiments, the first cell growth substrate), whereby removal of at least a portion of the second cell growth substrate from the first cell growth substrate produces a region of the first cell growth substrate devoid of cells proximal to one or more regions of the first and/or second cell growth substrate populated by cells.

The first and/or second cell growth substrate can be uncoated or coated, e.g., with a cell adhesion or growth-promoting reagent, or with a test reagent whose effect on cell growth and/or migration is to be assessed in an in vitro wound healing assay. Thus, in one class of embodiments, the first and/or second cell growth substrate comprises a coating that includes one or more of: a positively charged polymer (e.g., polylysine or polyethyleneimine), an extracellular matrix component, a protein or other polypeptide, collagen, laminin, fibronectin, vitronectin, gelatin, an antibody (e.g., an anti-receptor or anti-integrin antibody), a lectin, or a receptor ligand (e.g., an integrin ligand, e.g., an RGD peptide).

The first cell growth substrate optionally includes reference marks that indicate the position(s) of the second cell growth substrate or the removable portion(s) thereof. In one embodiment, the first cell growth substrate comprises at least one reference mark which indicates the position on the first cell growth substrate of an edge of the second cell growth substrate or a removable portion thereof.

Kits including a device of the invention, packaged in one or more containers along with instructional materials, culture media, assay reagents, and/or the like, form another feature of the invention. One general class of embodiments provides a kit that includes a device comprising a first cell growth substrate and a second cell growth substrate disposed on the first cell growth substrate, at least a portion of the second cell growth substrate being removable from the first cell growth substrate, and at least a first culture medium, packaged in one or more containers. The kit optionally also includes a second culture medium. In one class of embodiments, the first culture medium has a calcium concentration less than 0.5 mM or less than 0.2 mM. The second culture medium optionally has a calcium concentration that is greater than that of the first culture medium (e.g., at least five times, at least ten times, or at least twenty times greater than that of the first culture medium).

Essentially all of the features noted above apply to this embodiment as well, as relevant, e.g., for composition and/or configuration of first and/or second cell growth substrates, coatings, and the like.

Systems including a device of the invention and one or more components such as a camera, a microscope, and/or a fluid handling element (e.g., an element configured to dispense fluid, e.g., culture medium, cells in suspension, test reagents, or the like, onto the first and/or second cell growth substrates) are also a feature of the invention.

Another general class of embodiments provides methods for modeling wounds in vitro. In the methods, a device comprising a first cell growth substrate and a second cell growth substrate disposed on the first cell growth substrate is provided. A confluent layer of cells is disposed on the second cell growth substrate (and, in certain embodiments, optionally also on the first cell growth substrate), and at least a portion of the second cell growth substrate is removed to create a model wound in the confluent layer of cells.

In one class of embodiments, one or more cells are disposed on the first and/or second cell growth substrate, and the cells are cultured under conditions that permit growth of the cells to form the confluent layer. The cells are typically cultured in a first culture medium to form the confluent layer; in certain embodiments, the first culture medium has a calcium concentration less than 0.5 mM or less than 0.2 mM. The cells are optionally cultured in the first culture medium or in a second culture medium after removal of at least a portion of the second cell growth substrate. In one class of embodiments, the second culture medium has a calcium concentration that is greater than that of the first culture medium (e.g., at least five times, at least ten times, or at least twenty times that of the first culture medium).

The method can be used to create essentially any number of model wounds of essentially any shape or size. Thus, in one class of embodiments, two or more portions of the cell growth substrate are independently removed to create two or more model wounds in the confluent layer of cells.

In a preferred class of embodiments, the methods include monitoring repair of the model wound (e.g., photographically, microscopically, and/or the like, to assess cell migration, directional migration, proliferation, and/or closure of the wound over time). In a related class of embodiments, after removal of at least a portion of the second cell growth substrate, one or more assays are performed on cells populating the first and/or second cell growth substrate, e.g., to detect (qualitatively or quantitatively) the presence of a nucleic acid (e.g., an RNA, e.g., an mRNA), the presence of a protein, an activity of a protein, or post-translational modification of a protein.

In certain embodiments, the cells in the layer are contacted with a test reagent, e.g., before or after wounding, to assay the effect of the test reagent on repair of wound (e.g., the effect on cell migration, directional migration, and/or proliferation) and/or on cellular responses to wounding. To contact the cells with the test reagent, the first and/or second cell growth substrate can be coated with the test reagent. In other embodiments, the cells are contacted with the test reagent by contacting the cells with a solution comprising the test reagent (e.g., by introducing the test reagent into the culture medium).

Essentially all of the features noted above apply to this embodiment as well, as relevant, e.g., for composition and/or configuration of first and/or second cell growth substrates, coatings, and the like.

Methods for making devices of the invention form another feature of the invention. Thus, one general class of embodiments provides methods of producing a device. In the methods, a first cell growth substrate is provided, and a second cell growth substrate is disposed on the first cell growth substrate. In one class of embodiments, the second cell growth substrate is disposed on the first cell growth substrate by electron beam lithography, photolithography, microelastomeric stamping, reactive ion etching, masking, or a combination thereof. The methods optionally include making at least one reference mark on a surface of the first cell growth substrate, the reference mark indicating the position on the first cell growth substrate of an edge of the second cell growth substrate or a removable portion thereof. The methods can include disposing a coating on the first and/or second cell growth substrate.

### BRIEF DESCRIPTION OF THE DRAWINGS

FIG. 1 schematically depicts a device for modeling wounds in vitro. Panel A is a cross-section of the device prior to removal of the second cell growth substrate. Panel B is a top view of the device prior to removal of the second cell growth substrate. Panel C is a top view of the device immediately following removal of the second cell growth substrate. Panel D is a top view of the device at a later time point than Panel C.

FIG. 2 schematically depicts a device for modeling wounds in vitro. Panel A is a top view of the device. Panel B is a top view of the device including a confluent cell layer. Panel C is a top view of the device including cells, following removal of portions of the second cell growth substrate.

FIG. 3 schematically depicts a device for modeling wounds in vitro. Panel A is a top view of the device. Panel B is a top view of the device including a confluent cell layer. Panel C is a top view of the device including cells, following removal of the second cell growth substrate.

FIG. 4 schematically depicts a device for modeling wounds in vitro. Panel A is a cross-section of the device prior to removal of the second cell growth substrate. Panel B is a top view of the device prior to removal of the second cell growth substrate. Panel C is a top view of the device immediately following removal of the second cell growth substrate. Panel D is a top view of the device at a later time point than Panel C.

FIG. 5 presents photographs of a model wound produced by scratching a confluent cell layer, at 0 hours (Panel A) and 96 hours (Panel B) after wounding.

FIG. 6 presents photographs of a model wound produced by scratching a confluent cell layer (Panel A) and by removing a second cell growth substrate (Panel B).

FIG. 7 presents photographs of a model wound produced in a confluent cell layer by removing a second cell growth substrate, at 0 hours (Panel A), 12 hours (Panel B), 24 hours (Panel C), and 36 hours (Panel D) after wounding.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

The term “about” as used herein indicates the value of a given quantity varies by +/−10% of the value, or optionally +/−5% of the value, or in some embodiments, by +/−1% of the value so described.

A “biocompatible material” is a material that is non-toxic to cells and that is capable of supporting cell attachment and growth (e.g., in the presence of appropriate culture medium and the like).

A “confluent cell layer” is a layer of cells within which each cell is in contact with neighboring cells, such that there is substantially no space separating adjacent cells. A cell layer is typically confluent over a certain area of interest, e.g., a tissue culture dish or a portion thereof, a well of a multiwell tissue culture plate or a portion thereof, or a tissue culture slide or a portion thereof. In certain embodiments, the confluent cell layer is a confluent cell monolayer; that is, a confluent cell layer that is one cell thick.

A variety of additional terms are defined or otherwise characterized herein.

### DETAILED DESCRIPTION

Devices for creating model wounds, e.g., in a confluent cell layer or a tissue or organ culture, are provided. Methods for simply and reproducibly creating model wounds are also described. These devices and methods are useful, for example, in in vitro wound healing assays of various types. Kits and systems related to the devices are also described, as are methods of producing such devices.

Thus, a first general class of embodiments provides a device for modeling wounds in vitro. The device includes a first cell growth substrate and a second cell growth substrate. The second cell growth substrate is disposed on the first cell growth substrate, and at least a portion of the second cell growth substrate is removable from the first cell growth substrate.

A number of cell growth surfaces and devices (many of which are commercially available) are well known in the art and can be adapted to the practice of the present invention. For example, in one class of embodiments, the first cell growth substrate is a tissue culture dish (sometimes referred to in the literature as a tissue culture plate; e.g., a circular, oblong, or rectangular dish, typically having a growth area of about 1 cm² to about 200 cm² or more, e.g., a 35 mm, 60 mm, 100 mm, or 150 mm tissue culture plate). In a related class of embodiments, the first cell growth substrate is a well of a multiwell tissue culture plate (e.g., a plate having from two to 96 or more wells). In another class of embodiments, the first cell growth substrate is a tissue culture slide or similar planar surface. In yet another class of embodiments, the first cell growth substrate comprises a non-planar surface.

In a preferred class of embodiments, the first cell growth substrate comprises (e.g., consists of) a biocompatible material. For example, the first cell growth substrate can comprise or consist of polystyrene, polypropylene, polycarbonate, or another biocompatible polymer. Alternatively, the first cell growth substrate can comprise essentially any material with a biocompatible coating to permit cell growth. For example, in one embodiment, the first cell growth substrate comprises glass coated with a biocompatible material (e.g., an extracellular matrix component or other substance that promotes cell attachment and growth). As initially provided, the first cell growth substrate is typically sterile or capable of being sterilized, e.g., by heat, radiation, chemical treatment, or the like. The first cell growth substrate is typically, but need not be, rigid or semi-rigid.

Similarly, the second cell growth substrate preferably comprises or consists of a biocompatible material (e.g., a biocompatible plastic, or essentially any material covered with a biocompatible coating). For example, in one class of embodiments, the second cell growth substrate comprises or consists of a plastic or polymer film. The second cell growth substrate is typically flexible, but can be, e.g., slightly flexible, semi-rigid, or rigid. As initially provided, the second cell growth substrate is typically sterile or capable of being sterilized, e.g., by heat, radiation, chemical treatment, or the like.

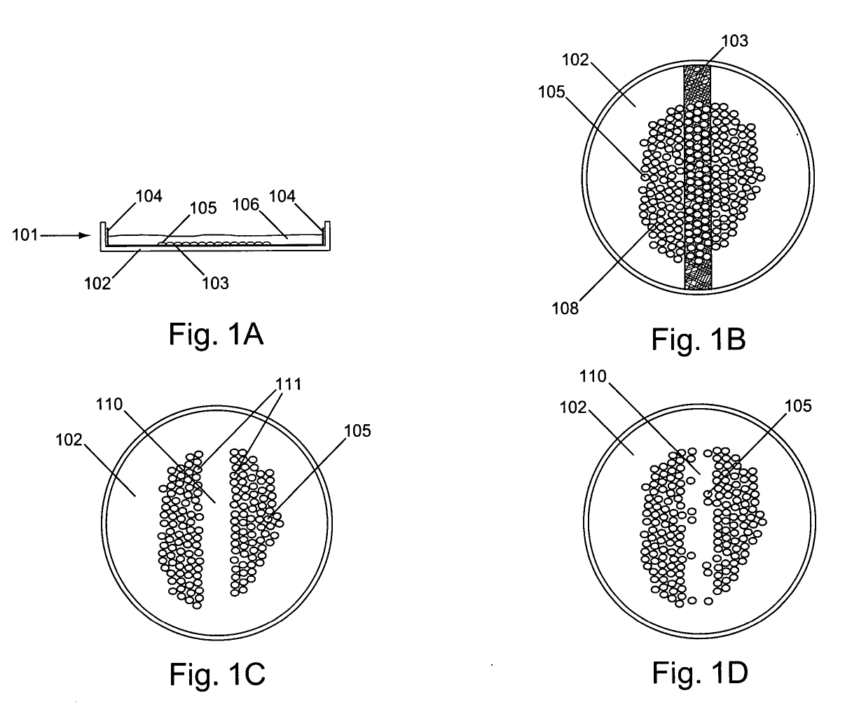

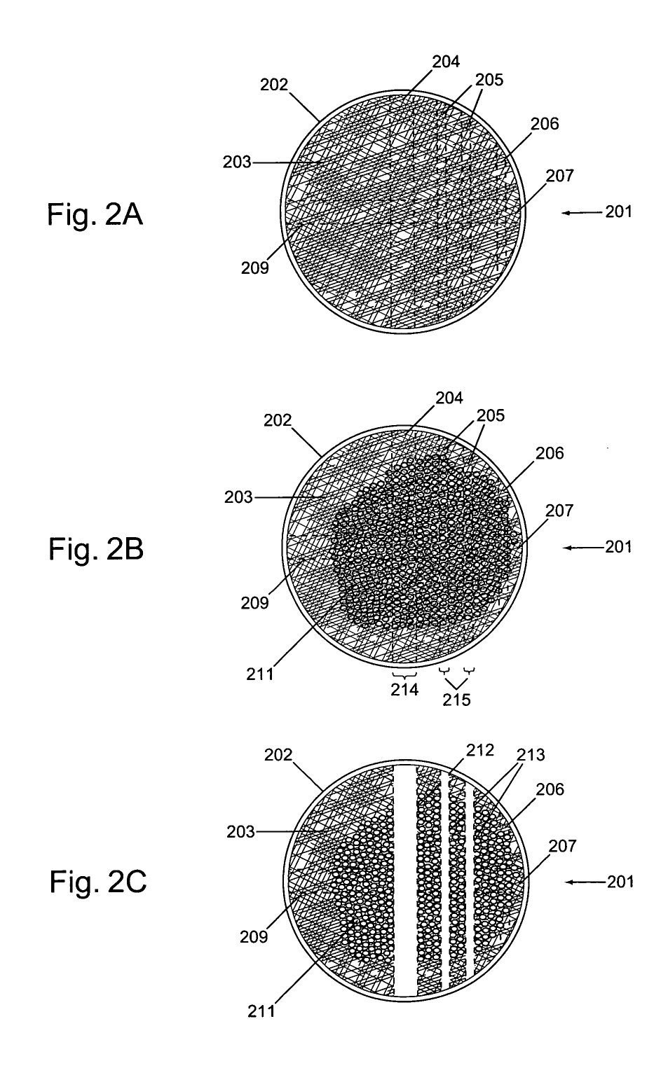

In one class of embodiments, the entire second cell growth substrate is removable from the first cell growth substrate (see, e.g., FIG. 1 Panels A and B). In another class of embodiments, a first portion of the second cell growth substrate is removable from the first cell growth substrate. A second portion of the second cell growth substrate remains disposed on the first cell growth substrate upon removal of the first portion. In certain embodiments, the second cell growth substrate comprises a tearable material. In one class of example embodiments, the second cell growth substrate comprises a plastic or polymer film that is perforated in a preselected pattern. The pattern delineates at least one portion of the second cell growth substrate to be removed (e.g., one, two, three, or more portions; see, e.g., FIG. 2 Panel A).

Depending, e.g., on the configuration of the portion(s) of the second cell growth substrate to be removed, the device can be used to create one or more model wounds, of the same or different sizes, in essentially any desired pattern. For example, in one class of embodiments, two or more (e.g., three, four, or more) portions of the second cell growth substrate are independently removable from the first cell growth substrate. Thus, one portion can be removed to create a single model wound, or two (or more) portions can be removed simultaneously or sequentially to create two (or more) model wounds. The portions can be of the same size (e.g., same width) and/or different sizes (e.g., different widths), permitting the creation of wounds of the same and/or different sizes.

As noted, the device is useful for creating model wounds, e.g., by disrupting a confluent layer of cells, a cell layer in a tissue or organ culture, or the like. Thus, in one class of embodiments, the device includes a confluent layer (e.g., a confluent monolayer) of cells disposed on the second cell growth substrate (and, in certain embodiments, also on the first cell growth substrate), whereby removal of at least a portion of the second cell growth substrate from the first cell growth substrate creates a model wound in the confluent layer of cells. It is worth noting that only a portion of the second (optionally also the first) cell growth substrate need be covered by the confluent cell layer; the entire available surface of the second (and optionally also the first) cell growth substrate need not be occupied by the cell layer.

In a related class of embodiments, the device comprises a confluent layer (e.g., a confluent monolayer) of cells disposed on the second cell growth substrate (and, in certain embodiments, the first cell growth substrate), whereby removal of at least a portion of the second cell growth substrate from the first cell growth substrate produces a region of the first cell growth substrate devoid of cells proximal to one or more regions of the first and/or second cell growth substrate populated by cells. See, e.g., FIG. 1 Panel C and FIG. 2 Panel C. In other embodiments, an organ or tissue layer can be disposed on the second (and optionally also the first) cell growth substrate.

The first and/or second cell growth substrate can be uncoated or coated, e.g., with a cell adhesion or growth-promoting reagent, or with a test reagent whose effect on cell growth and/or migration is to be assessed in an in vitro wound healing assay. Thus, in one class of embodiments, the first and/or second cell growth substrate comprises a coating that includes one or more of: a positively charged polymer (e.g., polylysine (D or L) or polyethyleneimine), an extracellular matrix component, a protein (including, e.g., a wild-type, truncated, mutant, modified, or chimeric protein) or other polypeptide, collagen, laminin, fibronectin, vitronectin, gelatin, an antibody (e.g., an anti-receptor or anti-integrin antibody), a receptor ligand (e.g., an integrin ligand, e.g., an RGD peptide), a lectin (e.g., concanavalin A) or other cross-linking agent, a drug, a specific inhibitor of a protein or protein family (e.g., a kinase or phosphatase inhibitor), a nucleic acid (e.g., an interfering RNA, e.g., an siRNA), or a receptor agonist or antagonist (e.g., an integrin agonist or antagonist). See, e.g., Bledi et al. (2000) Brain Res Brain Res Protoc. 5:282-9 and Rogers et al. (2002) J Cell Biol. 158:873-84, among many other examples of coated surfaces.

The second cell growth substrate or each removable portion thereof optionally includes a tab or is otherwise configured to permit a user of the device to conveniently grasp and remove the second cell growth substrate or portion thereof.

The initial position of the second cell growth substrate, or a removable portion thereof, is optionally indicated on the first cell growth substrate by one or more reference marks. Such marks permit a user of the device to precisely locate the initial borders of the wound created by removing the second cell growth substrate or portion thereof, even after cells adjacent to the wound have migrated and/or proliferated to infiltrate the initial wound site. Thus, in one class of embodiments, the first cell growth substrate comprises at least one reference mark which indicates the position on the first cell growth substrate of an edge of the second cell growth substrate or a removable portion thereof.

One class of example embodiments is illustrated in FIG. 1, Panels A-D. In this class of embodiments, device 101 includes second cell growth substrate 103 disposed on first cell growth substrate 102. As illustrated, first cell growth substrate 102 is a strip of biocompatible material. It is worth noting that two or more such strips, of essentially any size and shape, are optionally disposed on first cell growth substrate 102 in essentially any configuration. As illustrated in Panels A and B, a confluent layer 108 of cells 105 is disposed on second cell growth substrate 103 and also on first cell growth substrate 102. Cells 105 are surrounded by culture medium 106. For ease of removal of second cell growth substrate 103 from first cell growth substrate 102, second cell growth substrate 103 ends in tabs 104, which are positioned above the surface of culture medium 106. Either tab 104 can, e.g., be grasped with sterile forceps to remove second cell growth substrate 103 from first cell growth substrate 102, producing model wound 110 (region 110 of first cell growth substrate 102 devoid of cells, proximal to regions 111 populated by cells; Panel C). Region 110 is then monitored, e.g., microscopically or photographically, over time, for example, to assess migration and/or proliferation of cells 105 from regions 111 to heal the model wound (e.g., Panel D).

Another class of embodiments is illustrated in FIG. 2 Panels A-C. In this class of embodiments, device 201 includes second cell growth substrate 203 disposed on first cell growth substrate 202. As illustrated, second cell growth substrate 203 is a film (e.g., a plastic or polymer film) that is perforated in a preselected pattern. Perforations 209 (dashed lines) delineate portions 204, 205, 206, and 207 (Panel A). Portions 204-206 are independently removable from first cell growth substrate 202, such that one or more of these portions can be removed while the remainder of second cell growth substrate 203 remains disposed on first cell growth substrate 202, as illustrated in Panel C, in which portions 204 and 205 have been removed. It will be evident that two intersecting portions of the second cell growth substrate (e.g., 204 and 207) can be removed to create a cross-shaped wound, or the like. As illustrated, the two portions 205 have the same width 215, while the other portions have different widths (e.g., width 214 of portion 204 is greater than width 215). It will be evident that the removable portion(s) of second cell growth substrate 203 can be essentially any desired size and shape and can be configured in essentially any desired pattern, to form essentially any number of wounds of any configuration. For example, each portion can be rectangular and its width can be varied as desired, e.g., between 0.25 mm (or less) and 1.5 mm (or more). As in the example described above, the device can include cells, e.g. a confluent layer of cells 211 disposed on second cell growth substrate 203 (Panel B). Removal of at least one portion of second cell growth substrate 203 produces at least one model wound; for example, as shown in Panel C, removal of portions 204 and 205 produces model wounds 212 and 213. As noted, regions 212 and 213 can be monitored microscopically and/or photographically, to follow healing of the model wounds. As another example, alternatively or in addition, the cells can be removed from the first and/or second cell growth substrates, lysed, and subjected to biochemical assays (e.g., for nucleic acid transcription and/or translation, post-translational modification and/or activity of a protein, or the like) to investigate cellular responses to wounding. For example, up- or down-regulation of nucleic acid transcription, up- or down-regulation of protein expression or activity, change in post-translational modification of a protein (e.g., phosphorylation, lipidation, myristoylation, methylation, ubiquitination, glycosylation, and the like), change in second messenger concentration, or the like, can be monitored over time in response to wounding. Removable portions of the second cell growth substrate optionally comprise tabs to assist in their removal, e.g., with sterile forceps.

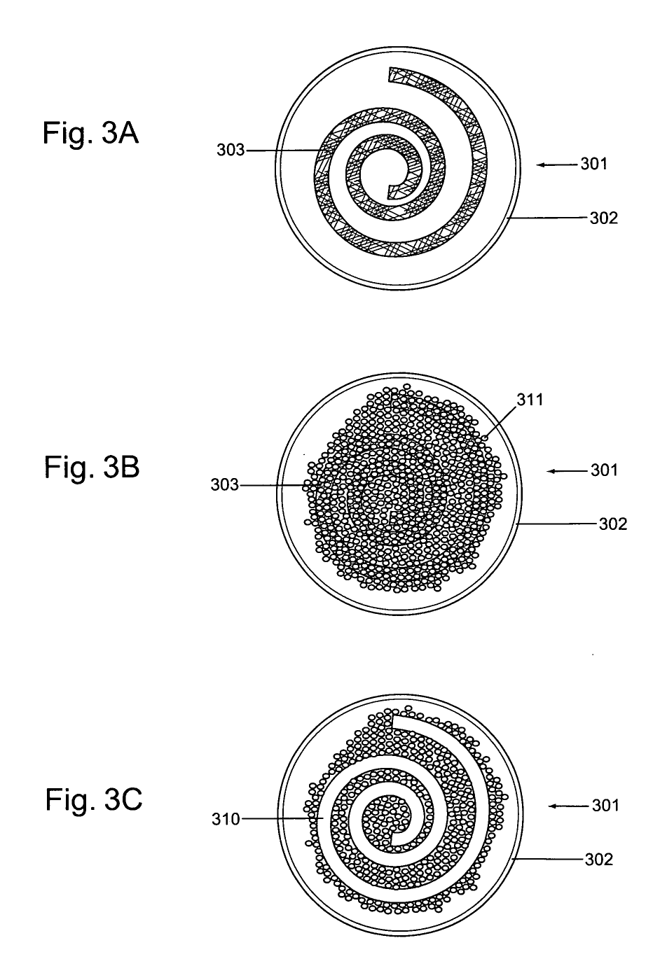

In the embodiments illustrated in FIGS. 1 and 2, each second cell growth substrate or removable portion thereof is substantially rectangular and has a length significantly greater than its width. However, it is worth noting that the second cell growth substrate or the removable portion(s) thereof can be of essentially any desired size and/or shape, e.g., regular or irregular, circular, polygonal, or the like. For example, in the embodiment illustrated in FIG. 3, device 301 includes first cell growth substrate 302 and second cell growth substrate 303. Second cell growth substrate 303 is disposed on first cell growth substrate 302 in a spiral track (Panel A). Growth of confluent cell layer 311 (Panel B) and removal of second cell growth substrate 303 thus creates spiral-shaped wound 310 in the cell layer, conveniently maximizing the area of wounded relative to unwounded cells (Panel C; compare with FIG. 1 Panel C). Maximizing the wound area can, e.g., increase signal intensity in assays for the presence, modification state, and/or activity of one or more cellular components, e.g., in cells harvested from first cell growth substrate 302 at one or more time points after removal of second cell growth substrate 303.

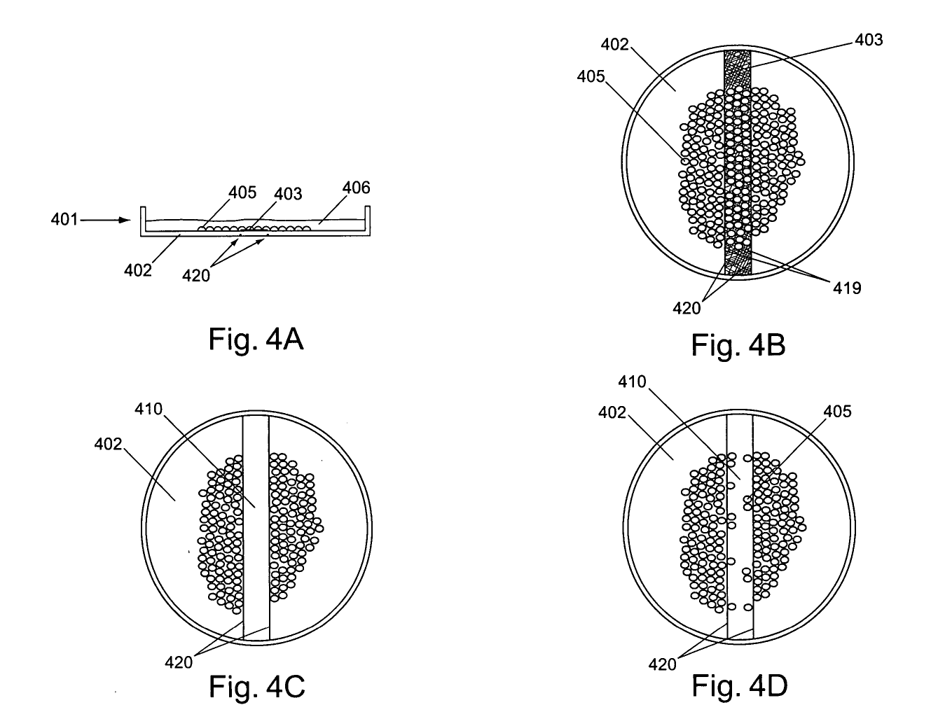

Yet another class of example embodiments is illustrated in FIG. 4, Panels A-D. This class of embodiments is similar to that illustrated in FIG. 1, with the addition of reference marks indicating the initial position of the second cell growth substrate on the first cell growth substrate. In this class of embodiments, device 401 includes second cell growth substrate 403 disposed on first cell growth substrate 402. As illustrated in Panels A and B, a confluent layer of cells 405 is disposed on second cell growth substrate 403 and also on first cell growth substrate 402 and is surrounded by culture medium 406. The position of second cell growth substrate 403 on first cell growth substrate 402 is indicated by reference marks 420, which are congruent with edges 419 of second cell growth substrate 403. Removal of second cell growth substrate 403 produces model wound 410 (Panel C). The initial position of the second cell growth substrate, and thus of the initial wound, is indicated by reference marks 420 even after cells 405 have migrated and/or proliferated and begun to heal the wound (Panel D). The reference marks can, e.g., be made on the top surface of the first cell growth substrate or formed into the first cell growth substrate, or, as illustrated in FIG. 4, can be made on a bottom surface of the first cell growth substrate (e.g., on the underside of a tissue culture plate, well, or slide, such that a user of the device can locate the marks by focusing in a lower plane than that occupied by the cells).

In one aspect, a system including a device of the invention and one or more components such as a camera, a microscope, and/or a fluid handling element (e.g., an element configured to dispense fluid, e.g., culture medium, cells in suspension, test reagents, or the like, onto the first and/or second cell growth substrates) is a feature of the invention.

Another aspect of the invention provides methods for modeling wounds in vitro. In the methods, a device comprising a first cell growth substrate and a second cell growth substrate disposed on the first cell growth substrate is provided. A confluent layer (e.g., a confluent monolayer) of cells is disposed on the second cell growth substrate (and, in certain embodiments, optionally also on the first cell growth substrate), and at least a portion of the second cell growth substrate is removed to create a model wound in the confluent layer of cells. The device can be, e.g., any of those described herein. It is worth noting that only a portion of the second (optionally also the first) cell growth substrate need be covered by the confluent cell layer; the entire available surface of the second (and optionally also the first) cell growth substrate need not be occupied by the cell layer.

In one class of embodiments, one or more cells are disposed on the first and/or second cell growth substrate, and the cells are cultured under conditions that permit growth of the cells to form the confluent layer, e.g., surrounding at least part of the second cell growth substrate or removable portion thereof. The cells are typically cultured in a first culture medium to form the confluent layer. The first culture medium can be, e.g., a standard or custom medium. In certain embodiments, the first culture medium has a calcium concentration that is markedly reduced as compared to standard culture medium preparations. For example, standard preparations of Dulbecco's MEM (DMEM) supplemented with 10% fetal calf serum (FCS) have a calcium concentration of approximately 1.8 mM. In contrast, the first culture medium can have a calcium concentration less than 1.8 mM (e.g., less than 1.5 mM, less than 1 mM, less than 0.5 mM, or less than 0.2 mM). For example, the first culture medium can have a calcium concentration between 0.06 and 0.2 mM, which can be achieved, e.g., by using calcium-free DMEM, supplemented with 10% FCS that has been treated with Chelex™ (e.g., Chelex 100, analytical grade 100-200 mesh from Bio-Rad, Hercules, Calif.) to remove calcium, with the resultant calcium concentration of the final product brought to the desired concentration by addition of sterile CaCl₂ solution. The first culture medium is typically selected to be optimal for the cell type of interest; for example, for keratinocytes, the culture medium can be Epilife™ (Cascade Biologics, Portland, Oreg.; supplied with a calcium concentration of 60 μM) or a serum-free formulation based on Medium 154 (Cascade Biologics, Portland, Oreg.), supplemented with growth factors, and with a final calcium concentration of 0.05-0.2 mM. Use of a low calcium first medium can be advantageous, since it can, for example, prevent the formation of calcium-dependent cell-cell adhesions (e.g. cadherin or desmosomal mediated) and can thus prevent trauma to cells when the second cell growth substrate or portion thereof is removed, removing the section of cells adherent to it from their neighboring cells that remain on the first and/or second cell growth substrate. Without this modification, cells removed when the second growth substrate is lifted can rip away from their neighbors, leaving torn cell edges and a ragged, uneven cell wound border. The cells can be cultured in the first culture medium after removal of at least a portion of the second cell growth substrate, or they can be cultured in a second culture medium with the same or with a different calcium concentration as the first culture medium. In one class of embodiments, the second culture medium has a calcium concentration that is greater than that of the first culture medium (e.g., at least five times, at least ten times, or at least twenty times that of the first culture medium). For example, the second culture medium optionally applied after the culture has been wounded and in which the wound healing or other assay is optionally performed can have a higher, standard calcium concentration, e.g., of between 1 and 1.8 mM. Typically, the second culture medium is also selected to be optimal for the cell type of interest.

The method can be used to create essentially any number of model wounds of essentially any shape or size. Thus, in one class of embodiments, two or more portions of the cell growth substrate are independently removed to create two or more model wounds in the confluent layer of cells. It will be evident that, in other embodiments, two intersecting portions of the second cell growth substrate can be removed to create a single, cross-shaped wound, or the like.

In a preferred class of embodiments, the methods include monitoring repair of the model wound (e.g., photographically, microscopically, and/or the like, to assess cell migration, directional migration, proliferation, and/or closure of the wound over time). For example, cell migration in the absence of proliferation can be monitored by incubating the cells with a proliferation inhibitor (such as, e.g., mitomycin C), typically prior to removing the second cell growth substrate or portion thereof to create the wound.

In a related class of embodiments, cells adjacent to the wound are subjected to one or more assays to determine the presence, modification state (e.g., phosphorylation state of a protein), and/or activity of one or more cellular components (e.g., one or more proteins, nucleic acids, lipids, second messengers, and/or the like), for example, to investigate cellular responses to wounding. The expression, modification state, or activity of the one or more cellular components in cells adjacent to the wound is optionally compared to that in cells more distal to the wound (or from an unwounded cell layer, or the like), to assess changes in nucleic acid or protein expression, post-translational modification of proteins, second messenger concentration, or the like. Thus, in one class of embodiments, one or more assays are performed on cells populating the first and/or second cell growth substrate, e.g., at one or more preselected time points after removal of at least one portion of the second cell growth substrate. The assay(s) can, for example, detect presence of a nucleic acid (e.g., an RNA, e.g., an mRNA), presence of a protein, activity of a protein (e.g., an enzymatic or other activity), and/or post-translational modification of a protein (including, but not limited to, phosphorylation, ubiquitination, lipidation, myristoylation, glycosylation, and methylation). Such detection can be quantitative or qualitative.

In certain embodiments, the cells in the layer are contacted with a test reagent, e.g., before or after wounding, to assay the effect of the test reagent on repair of wound (e.g., the effect on cell migration, directional migration, and/or proliferation) and/or on cellular responses to wounding. To contact the cells with the test reagent, the first and/or second cell growth substrate can be coated with the test reagent. Typically, this coating is performed prior to disposing the confluent layer of cells on the second cell growth substrate. In other embodiments, the cells are contacted with the test reagent by contacting the cells with a solution comprising the test reagent (e.g., by introducing the test reagent into the culture medium). Test reagents include, but are not limited to, an extracellular matrix component, a protein (including, e.g., a wild-type, truncated, mutant, modified, or chimeric protein) or other polypeptide, collagen, laminin, fibronectin, vitronectin, gelatin, an antibody (e.g., an anti-receptor or anti-integrin antibody), a receptor ligand (e.g., an integrin ligand, e.g., an RGD peptide), a lectin (e.g., concanavalin A) or other cross-linking agent, a drug, a specific inhibitor of a protein (or protein family), a nucleic acid (e.g., an interfering RNA, e.g., an siRNA), or a receptor agonist or antagonist (e.g., an integrin agonist or antagonist).

The methods of the present invention offer a number of advantages. For example, the methods result in wounds having more uniform width along each wound and between wounds. The wounds can be of preselected width and can be in essentially any desired configuration. Minimal (or no) damage is inflicted on cells adjacent to the wound, resulting in more reproducible wound healing.

The cells used in the methods and devices can be of essentially any type, including, but not limited to, mammalian cells (including, but not limited to, human, mouse, rat, monkey, or hamster cells), keratinocytes, fibroblasts, epithelial cells, endothelial cells, cultured cells, primary cultured cells, immortalized cell lines, plant cells, and the like.

Kits including a device of the invention, packaged in one or more containers along with instructional materials, culture media, assay reagents, and/or the like, form another feature of the invention. One general class of embodiments provides a kit that includes a device comprising a first cell growth substrate and a second cell growth substrate disposed on the first cell growth substrate, at least a portion of the second cell growth substrate being removable from the first cell growth substrate, and at least a first culture medium, packaged in one or more containers. The kit can also include a second (third, fourth, etc.) culture medium.

The first culture medium can be, e.g., a standard or custom medium. In certain embodiments, the first culture medium has a calcium concentration that is markedly reduced as compared to standard culture medium preparations. For example, standard preparations of Dulbecco's MEM (DMEM) supplemented with 10% fetal calf serum (FCS) have a calcium concentration of approximately 1.8 mM. In contrast, the first culture medium can have a calcium concentration less than 1.8 mM (e.g., less than 1.5 mM, less than 1 mM, less than 0.5 mM, or less than 0.2 mM). For example, the first culture medium can have a calcium concentration between 0.06 and 0.2 mM, which can be achieved, e.g., by using calcium-free DMEM, supplemented with 10% FCS that has been Chelex™ treated to remove calcium, with the resultant calcium concentration of the final product brought to the desired concentration by addition of sterile CaCl₂ solution. The first culture medium is typically selected to be optimal for the cell type of interest to a user of the kit; for example, for keratinocytes, the culture medium can be Epilife™ or a serum-free formulation based on Medium 154, supplemented with growth factors, and with a final calcium concentration of 0.05-0.2 mM. Use of a low calcium first medium for growing a confluent cell layer on the first and/or second cell growth substrates can be advantageous in producing cleaner edged wounds and preventing cell damage, as noted above. The second culture medium, when present, can have the same or a different calcium concentration as the first culture medium. In one class of embodiments, the second culture medium has a calcium concentration that is greater than that of the first culture medium (e.g., at least five times, at least ten times, or at least twenty times greater than that of the first culture medium). For example, the second culture medium, which is optionally applied after the culture has been wounded, can have a higher, standard calcium concentration, e.g., of between 1 and 1.8 mM. Typically, the second culture medium is also selected to be optimal for the cell type of interest.

The kit can also include instructions, e.g., for using the kit to create model wounds, monitor healing of the wounds, and/or assay cellular responses to wounding, reagents for assaying cellular responses to wounding, and/or the like.

Methods for making devices of the invention form another feature of the invention. Thus, one general class of embodiments provides methods of producing a device. In the methods, a first cell growth substrate is provided, and a second cell growth substrate is disposed on the first cell growth substrate.

The second cell growth substrate can be applied to the first cell growth substrate using any of a variety of techniques known in the art. In certain embodiments, for example, the second cell growth substrate is disposed on the first cell growth substrate using one or more microfabrication techniques. Such techniques can allow the application of the second growth substrate (e.g., a biocompatible film) in very precise straight edged and very narrow (e.g., micron range) widths, to generate precisely edged and narrow wounds. For example, the width of the second cell growth substrate or removable portion thereof (and therefore the width of the resulting wound) can be less than about 3 mm, less than about 1 mm, less than about 500 μm, or even less than about 250 μm; e.g., the width can be between about 100 μm and about 1 mm. The advantage of the narrower wound made possible by microfabrication is the decrease in observation time required for measuring healing of said wound. The second growth substrate can be applied using any of a number of available microfabrication techniques, including, but not limited to electron beam lithography, photolithography, microelastomeric stamping, reactive ion etching, or masking. Such microfabrication techniques are known in the art and can readily be adapted for the practice of the present invention; see, e.g., Vijay Varadan (2004) Microfabrication Techniques for Polymeric Mems (Micro & Nanoscience & Technology), The Institute of Physics; Hierlemann et al. (2003) “Microfabrication Techniques for Chemical/Biosensors” Proc of the IEEE 91(6): 839-863; Vasile et al. (1999) “Microfabrication techniques using focused ion beams and emergent applications” Micron 30(3):235-244; Mark Madou Fundamentals of Microfabrication, CRC Press; Stephen D. Senturia (2000) Microsystem Design, Kluwer Academic Press; and Richard C. Jaeger (2002) Introduction to Microelectronic Fabrication, 2nd Edition, Addison-Wesley. The application pattern of the second growth substrate can, for example, be linear, or it can be a narrow spiral track that begins at the center of the first cell growth substrate and spirals outward (or vice versa).

The methods optionally include making at least one reference mark on a surface of the first cell growth substrate, the reference mark indicating the position on the first cell growth substrate of an edge of the second cell growth substrate or a removable portion thereof. For example, a microfabrication technique can be used to etch marks on a surface of the first cell growth substrate (e.g. the underside of a cell culture plate or slide) that are precisely congruent with the position of the overlying second cell growth substrate.

In one class of embodiments, the methods include disposing a coating on the first and/or second cell growth substrate (e.g., a biocompatible material, a cell adhesion or growth-promoting reagent, a test reagent, or the like, as noted herein).

### Cell Culture

Methods for culturing cells are established and well known in the art. Typically, cells are cultured in a standard medium, e.g., a standard commercial culture medium, such as Dulbecco's modified Eagle's medium supplemented with serum (e.g., 10% fetal bovine serum), or in serum free medium, under controlled humidity and CO₂ concentration suitable for maintaining neutral buffered pH (e.g., at pH between 7.0 and 7.2). Optionally, the medium contains growth factors and/or other growth-promoting agents, antibiotics to prevent bacterial growth, e.g., penicillin, streptomycin, etc., and/or additional nutrients, such as L-glutamine, sodium pyruvate, non-essential amino acids, additional supplements to promote favorable growth characteristics, e.g., trypsin, β-mercaptoethanol, and the like.

Procedures for maintaining cells, e.g., mammalian cells, in culture have been extensively reported and are known to those of skill in the art. General protocols are provided, e.g., in Freshney (1994) Culture of Animal Cells, a Manual of Basic Technique, third edition, Wiley-Liss, New York and the references cited therein; Paul (1975) Cell and Tissue Culture, 5^{th} ed., Livingston, Edinburgh; Adams (1980) Laboratory Techniques in Biochemistry and Molecular Biology-Cell Culture for Biochemists, Work and Burdon (eds.) Elsevier, Amsterdam; Atlas and Parks (eds) The Handbook of Microbiological Media (1993) CRC Press, Boca Raton, Fla.; Payne et al. (1992) Plant Cell and Tissue Culture in Liquid Systems John Wiley & Sons, Inc. New York, N.Y.; and Gamborg and Phillips (eds) (1995) Plant Cell, Tissue and Organ Culture; Fundamental Methods Springer Lab Manual, Springer-Verlag (Berlin Heidelberg New York). Additionally, variations in such procedures adapted to the present invention are readily determined through routine experimentation.

### Biochemical Assays

Assays for determining the presence, quantity, modification state, and/or activity levels of various cellular components (including, among many others, Northern, Southern, Western, and dot blots, immunoprecipitation, ELISA, rt-PCR, microarray analysis, radiolabeling, mass spectroscopy, various chromatography and electrophoretic techniques, and enzyme and other protein activity assays) are likewise extensively reported and known to those of skill in the art. See, e.g., Ausubel et al. Current Protocols in Molecular Biology (supplemented through 2004) John Wiley & Sons, New York; and Sambrook et al. Molecular Cloning—A Laboratory Manual (3rd Ed.), Vol. 1-3, Cold Spring Harbor Laboratory, Cold Spring Harbor, N.Y., 2001.

### EXAMPLES

The following sets forth a series of experiments that demonstrate construction and use of a device for creating model wounds in vitro and an in vitro wounding method. It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

To construct an example device such as that illustrated in FIG. 1, Parafilm™ strips of 1 mm in width and 5 cm in length are used as an example second cell growth substrate. The strips are washed overnight in sterile PBS (phosphate-buffered saline) and then sterilized and dried by exposure to ultraviolet light for 1-2 hours. The strips are then applied to the bottom of a sterile 35 mm cell culture dish (see FIG. 1 Panels A and B). A sterile cell scraper is used to adhere the strips to the plastic dish (an example first cell growth substrate). Gentle pressure is applied to ensure that no residue will remain on the dish after removal of the Parafilm™ strip from the dish. Cells are then applied directly to the dish around the Parafilm™ and permitted to grow under appropriate conditions. Upon the cells reaching confluence (and preferably prior to differentiation and/or stacking of the cells, if applicable), the strips are removed from the dishes using sterile forceps, creating a wound.

The wounds generated in this way are uniform in width and size. There is minimal intra-wound and inter-wound variability and no damage along the edge of the wound. Wounds heal well within 48 hours of wounding.

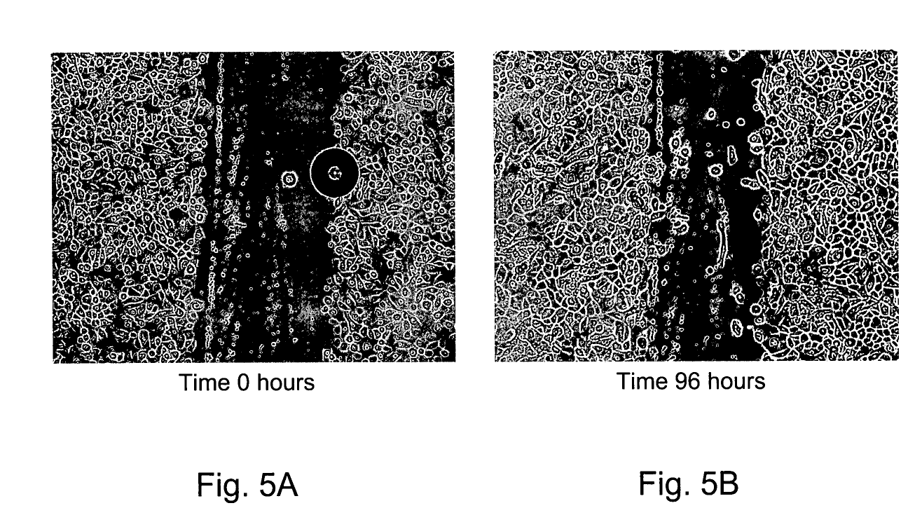

Photographs of a wound produced by scratching a confluent layer of cells are shown in FIG. 5, at 0 hours (Panel A) and 96 hours (Panel B) after wounding. Note that the wound has failed to heal after as long as 96 hours.

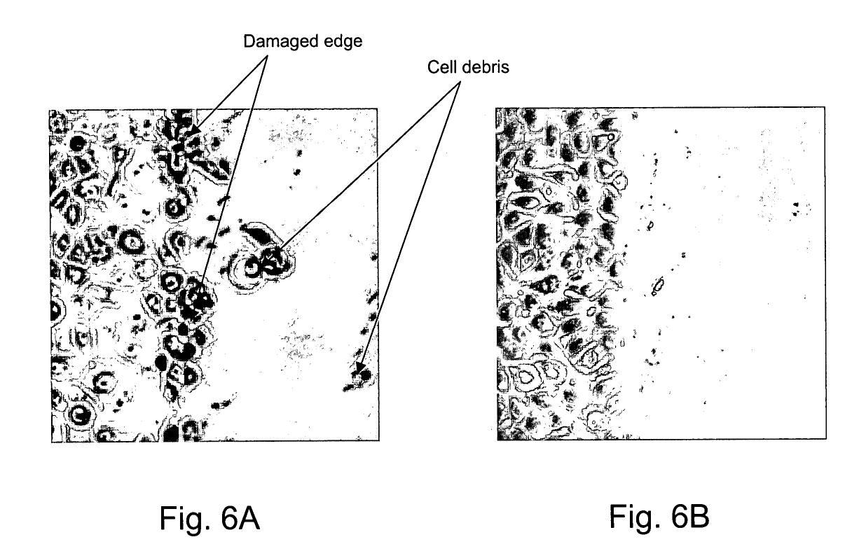

FIG. 6 presents photographs comparing model wounds produced by scratching a confluent cell layer (Panel A, note the damaged wound edge and cell debris visible) and by removal of a second cell growth substrate (Panel B, note the clean wound edge).

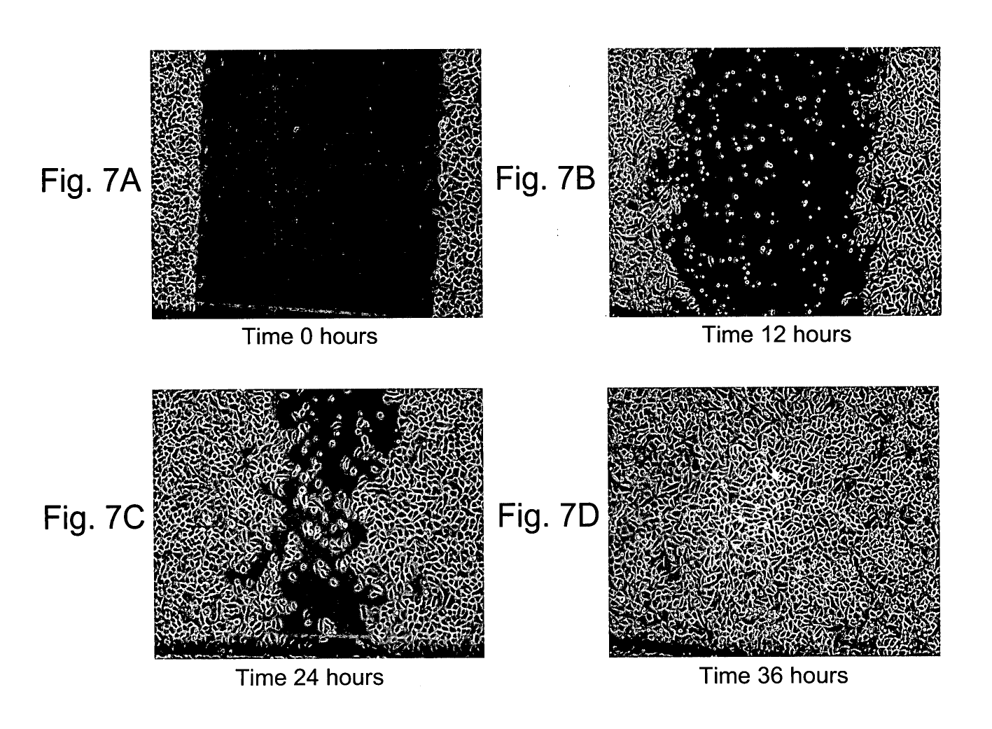

FIG. 7 presents photographs of a model wound produced in a confluent cell layer by removal of a second cell growth substrate, at 0 hours (Panel A), 12 hours (Panel B), 24 hours (Panel C), and 36 hours (Panel D) after wounding. The wound edge is clean at 0 hours, and the wound has healed well by 36 hours.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A device for modeling wounds in vitro, the device comprising:
a first cell growth substrate; and
a second cell growth substrate disposed on the first cell growth substrate, at least a portion of the second cell growth substrate being removable from the first cell growth substrate.

2. The device of claim 1, wherein the first cell growth substrate is a tissue culture dish or a well of a multiwell tissue culture plate.

3. The device of claim 1, wherein the first cell growth substrate is a tissue culture slide.

4. The device of claim 1, wherein the first cell growth substrate comprises a biocompatible material.

5. The device of claim 4, wherein the first cell growth substrate comprises polystyrene, polypropylene, or polycarbonate.

6. The device of claim 4, wherein the first cell growth substrate comprises glass coated with the biocompatible material.

7. The device of claim 1, wherein the second cell growth substrate comprises a biocompatible material.

8. The device of claim 7, wherein the second cell growth substrate comprises a plastic or polymer film.

9. The device of claim 1, wherein the entire second cell growth substrate is removable from the first cell growth substrate.

10. The device of claim 1, wherein the second cell growth substrate comprises a tearable material.

11. The device of claim 1, wherein the second cell growth substrate comprises a plastic or polymer film perforated in a preselected pattern, which pattern delineates at least one portion of the second cell growth substrate to be removed.

12. The device of claim 1, wherein a first portion of the second cell growth substrate is removable from the first cell growth substrate, a second portion of the second cell growth substrate remaining disposed on the first cell growth substrate upon removal of the first portion.

13. The device of claim 1, wherein two or more portions of the second cell growth substrate are independently removable from the first cell growth substrate.

14. The device of claim 13, wherein the two or more portions of the second cell growth substrate have different widths.

15. The device of claim 1, comprising a confluent layer of cells disposed on the second cell growth substrate, whereby removal of at least a portion of the second cell growth substrate from the first cell growth substrate creates a model wound in the confluent layer of cells.

16. The device of claim 1, comprising a confluent layer of cells disposed on the second cell growth substrate, whereby removal of at least a portion of the second cell growth substrate from the first cell growth substrate produces a region of the first cell growth substrate devoid of cells proximal to one or more regions of the first and/or second cell growth substrate populated by cells.

17. The device of claim 1, wherein the first and/or second cell growth substrate comprises a coating, the coating comprising one or more of: a positively charged polymer, polylysine, polyethyleneimine, an extracellular matrix component, a protein, collagen, laminin, fibronectin, vitronectin, gelatin, an antibody, a lectin, a receptor ligand, or an integrin ligand.

18. The device of claim 1, wherein the first cell growth substrate comprises at least one reference mark, which reference mark indicates the position on the first cell growth substrate of an edge of the second cell growth substrate or a removable portion thereof.

19. A kit, comprising:
a device comprising a first cell growth substrate and a second cell growth substrate disposed on the first cell growth substrate, at least a portion of the second cell growth substrate being removable from the first cell growth substrate; and
at least a first culture medium, packaged in one or more containers.

20. The kit of claim 19, wherein the first culture medium has a calcium concentration less than 0.5 mM or less than 0.2 mM.

21. The kit of claim 20, comprising a second culture medium, wherein the second culture medium has a calcium concentration that is greater than that of the first culture medium.

22. The kit of claim 21, wherein the calcium concentration of the second culture medium is at least five times, at least ten times, or at least twenty times that of the first culture medium.

23. A method for modeling wounds in vitro, the method comprising:
providing a device comprising a first cell growth substrate and a second cell growth substrate disposed on the first cell growth substrate;
disposing a confluent layer of cells on the second cell growth substrate; and
removing at least a portion of the second cell growth substrate, thereby creating a model wound in the confluent layer of cells.

24. The method of claim 23, wherein disposing a confluent layer of cells on the second cell growth substrate comprises disposing one or more cells on the first and/or second cell growth substrate and culturing the cells under conditions that permit growth of the cells to form the confluent layer.

25. The method of claim 24, wherein the cells are cultured in a first culture medium to form the confluent layer, the first culture medium having a calcium concentration less than 0.5 mM or less than 0.2 mM.

26. The method of claim 25, comprising culturing the cells in a second culture medium after removal of at least a portion of the second cell growth substrate, the second culture medium having a calcium concentration that is greater than that of the first culture medium.

27. The method of claim 26, wherein the calcium concentration of the second culture medium is at least five times, at least ten times, or at least twenty times that of the first culture medium.

28. The method of claim 23, wherein removing at least a portion of the second cell growth substrate comprises independently removing two or more portions of the second cell growth substrate, thereby creating two or more model wounds in the confluent layer of cells.

29. The method of claim 23, comprising monitoring repair of the model wound.

30. The method of claim 23, comprising performing one or more assays on cells populating the first and/or second cell growth substrates, after removing at least a portion of the second cell growth substrate.

31. The method of claim 30, wherein the one or more assays detect one or more of: presence of a nucleic acid, an RNA, or an mRNA; presence of a protein; activity of a protein; or post-translational modification of a protein.

32. The method of claim 23, comprising contacting the cells in the layer with a test reagent.

33. The method of claim 32, wherein contacting the cells with the test reagent comprises coating the first and/or second cell growth substrate with the test reagent.

34. The method of claim 32, wherein contacting the cells with the test reagent comprises contacting the cells with a solution comprising the test reagent.

35. A method of producing a device, the method comprising:
providing a first cell growth substrate; and
disposing a second cell growth substrate on the first cell growth substrate.

36. The method of claim 35, wherein the second cell growth substrate is disposed on the first cell growth substrate by electron beam lithography, photolithography, microelastomeric stamping, reactive ion etching, masking, or a combination thereof.

37. The method of claim 35, comprising making at least one reference mark on a surface of the first cell growth substrate, the reference mark indicating the position on the first cell growth substrate of an edge of the second cell growth substrate or a removable portion thereof.
